(19) United States
(12) Patent Application Publication
(10) Pub. No.: US 2022/0133138 A1
(43) Pub. Date: May 5, 2022

(54) DILATOR-LESS AND OBTURATOR-LESS INTRODUCER FOR VIEWING AND ACTING ON INTERNAL PASSAGEWAYS OR TISSUE

(71) Applicant: Clearmind Biomedical, Inc., San Jose, CA (US)

(72) Inventors: Sheng-chi Lin, Taipei (TW); Feng-cheng Chang, Taipei (TW); Yi-chen Wang, Taipei (TW)

(21) Appl. No.: 17/083,957

(22) Filed: Oct. 29, 2020

(51) Int. Cl. A61B 1/018; A61B 1/00; A61B 1/05; A61B 1/06; A61B 1/005; A61B 17/34

(52) U.S. Cl. CPC A61B 1/018; A61B 1/00137; A61B 1/05; A61B 1/0676; A61B 17/3421; A61B 1/00045; A61B 1/00096; A61B 1/00087; A61B 1/0052

(57) ABSTRACT

A medical device configured to move an introducer into a passageway or tissue without the conventional use of an obturator or dilator while concurrently affording visual control over the movement of a distal end of the introducer in the passageway or tissue.

### FIELD

This patent specification is in the field of methods and devices for viewing and acting on internal passageways and tissue.

### BACKGROUND

In certain medical procedures, a tubular introducer is inserted in patient tissue or in a patient's cavity or lumen to provide a channel for surgical instruments and/or an endoscope. Before inserting the introducer into the patient, a rod-like obturator is eased into the introducer until the obturator's tip protrudes distally from the introducer. So assembled, the introducer/obturator unit is pushed into a patient's tissue or a patient's cavity or lumen. If into tissue that must be cut for entry, an obturator with a sharp or bladed tip is used to help form a pathway for the introducer. If into a cavity or lumen or tissue that need not or should not be cut, the obturator tip may be blunt or a blunt dilator can be used to clear or enlarge the cavity or lumen or enter soft tissue. In each case, after the introducer has been inserted to the desired depth in the patient, the obturator or dilator is withdrawn to free the interior of the introducer for surgical instruments such as injection needles, abraders, scalpels, pincers, coagulators, or other instruments and/or of endoscopes for viewing internal tissue. The introducer with the obturator or dilator therein can be introduced into tissue under ultrasound guidance or guidance from other imaging systems. A trocar, which comprises a cannula and an obturator, is an alternative instrument. An example of trocars can be seen in U.S. Pat. No. 6,960,164, hereby incorporated by reference.

Trocar cannulas are often rigid and so are obturators or dilators that slide therein. The trocar's cannula outside diameter typically is several mm, such as 5-12 mm, and its internal diameter needs to allow a mm or so clearance for the passage of surgical instruments or viewing devices. Companies such as Storz, Wolf, and AESCULAP are known suppliers of trocars. Introducers or introducer sheaths can be thinner and more flexible. Companies such as Medtronic have offered introducer sheaths through which catheters can be inserted.

Intracranial medical procedures present unique challenges to accurately and safely place introducers in the brain and provide channels for surgical instruments or endoscopes. Some aspects of the new approaches described in this patent specification are particularly efficacious for intracranial procedures.

### SUMMARY OF DISCLOSURE

According to some embodiments, a method of endoscopic intervention using a dilator-less and obturator-less introducer comprises: securing an opening at a proximal end of an optical cap to a distal tip of an endoscope cannula that has distally facing camera and light source, and intermediate portion and a proximal portion; inserting the distal tip of the endoscope cannula, with said optical cap secured thereto, in the distal direction into a tubular introducer that has open proximal and distal ends such that: a distal end of the optical cap protrudes distally from the distal end of the introducer; an outside wall of a proximal end of said optical cap and an outside wall of the distal end of the introducer cannula are dimensioned to form a smooth transition from the introducer to the optical cap; and the proximal portion of the endoscope cannula extends proximally from the proximal end of the introducer and the intermediate portion of the endoscope cannula is between the proximal and distal ends of the introducer; inserting the introducer, with the intermediate portion of the endoscope cannula therein and with said optical cap secured to the distal end of the cannula, in the distal direction into a passageway or tissue, using the rounded distal end of said optical cap to dilate the passageway or form a passageway in tissue, while observing at a display images taken by said camera at the endoscope distal end through said optical cap; withdrawing the endoscope cannula together with the optical cap from the introducer in the proximal direction; and inserting one or more surgical instruments or devices distally into the introducer to act on tissue adjacent the distal end of the introducer.

According to some embodiments, the method further includes one or more of the following: inserting an introducer made of a flexible plastic material reinforced with a metal coil; inserting an introducer in which a distal portion of the introducer is free of said metal coil and is less rigid and more flexible than another portion of the introducer; selectively bending the introducer to deviate from a straight longitudinal axis; making at least a selected portion of a length of the introducer flexible and introducing a bent endoscope cannula therein to thus bend the introducer; inserting an introducer with a distal end that is beveled such that the diameter of its outside wall approaches the diameter of its inside wall toward a distal tip of said introducer; inserting an introducer having one or more collapsed lumens at an outside wall of the introducer; and introducing an implement into and thereby expanding one or more of said one or more lumens.

According to some embodiments, a medical device comprises: a tubular introducer having open proximal and distal ends; an endoscope cannula having a proximal end extending proximally from said introducer, an intermediate portion within said introducer, and a distal end at or protruding distally from the distal end of the introducer; said endoscope cannula having a camera and a light source at its distal end; an optical cap removably secured to said distal end of the endoscope and having a rounded distal end and a hollow proximal portion into which the distal end of the endoscope is inserted; wherein: said rounded distal end of the optical cap protrudes distally from the distal end of the introducer; and a distal portion of the introducer has an outside dimension that decreases toward the distal end of the introducer and the proximal portion of the optical cap has an outside dimension that matches an inside diameter of the distal end of the introducer to thereby provide a smooth transition between the outside of the optical cap and the outside of the distal end of the introducer; and an image display coupled to said camera to receive and display images taken with said camera through said optical cap; said introducer and cannula therein being configured for introduction as a unit into a patient cavity or tissue without an obturator or dilator.

According to some embodiments, the medical device further comprises one or more of the following: the introducer is made of a flexible tubular plastic material and an internal coil of a different material stiffening the tubular plastic material while still allowing at least a distal portion thereof to bend while or after being introduced into said passageway or tissue; the endoscope cannula includes a bend in its intermediate portion and said introducer is configured to bend to match the bend in the endoscope cannula; an endoscope handle from which said endoscope cannula extends distally, wherein said cannula is configured to bend at an intermediate portion under operator control applied at the endoscope handle, while the endoscope cannula is in the introducer; the endoscope cannula, optical cap, and introducer are configured for withdrawal of the endoscope together with the optical cap from the introducer in the proximal direction; and the optical cap is made of an optically transparent material.

According to some embodiments, a device comprises: a tubular introducer open at proximal and distal ends thereof; an elongated cannula having a camera and a light source at a distal end thereof and further having an intermediate portion that is inside said introducer, a proximal portion extending proximally from the introducer, and a distal portion; an optical cap secured to said distal portion of the cannula, over said camera and light source, and having a rounded end extending distally from the distal end of the introducer; wherein said cap and introducer are dimensioned to form a smooth transition from the distal end of the introducer to a proximal portion of the cap that extends distally from the introducer; and a display operatively coupled with said camera at the distal end of the cannula to receive images taken with the camera and display said images; said device being configured for insertion of the introducer, cannula, and optical cap into a passageway or tissue without using a dilator or obturator to form or dilate the passageway or tissue.

According to some embodiments, the device described in the preceding paragraph further includes one or more of the following: a distal portion of the introducer is more flexible than another portion of the introducer to thereby facilitate bending said distal portion as the introducer is being moved into a passageway or tissue; at least a portion of said introducer is made of a plastic material reinforced with a metal coil; at least a portion of said introducer is made of plural layers of plastic material configured to make a distal portion of the introducer less rigid and more flexible than another portion of the introducer; the device further includes at least one collapsed lumen secured to the outside of the introducer and configured to expand as a surgical device is introduced and moved distally therein along the length of the introducer; and one of the optical cap and the distal portion of the cannula includes a clip and the other includes a catch, configured for the clip to engage the catch and thereby secure the cap to the cannula.

### BRIEF DESCRIPTION OF THE DRAWINGS

FIGS. 1a-1d illustrate a medical device according to some embodiments where: FIG. 1a is a perspective view of an introducer, a viewing endoscope having a cannula inserted in the introducer, and an optical cap over a camera at a distal tip of the cannula; FIG. 1b is a partial longitudinal sectional view of the distal end of the device; FIG. 1c is a longitudinal sectional view of an intermediate portion of the device; and FIG. 1d is a perspective view showing a cross-section of an intermediate portion of the device.

FIGS. 2a-2f illustrate a process of using the medical device of FIGS. 1a-1d as well as additional features of the device.

FIGS. 3a and 3b illustrate a device with expandable lumens at an outside wall of an introducer, which can allow introduction of additional surgical instruments without significantly increasing the overall diameter of the introducer when pushed into a patient.

FIGS. 4a and 4b illustrate a medical device with a side lumen in an embodiment; FIG. 4c-4e illustrate a medical device with a side lumen in another embodiment.

### DETAILED DESCRIPTION OF EXAMPLES

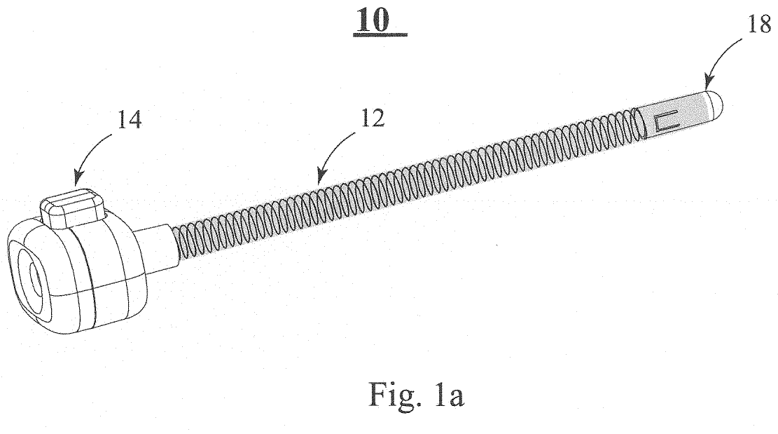

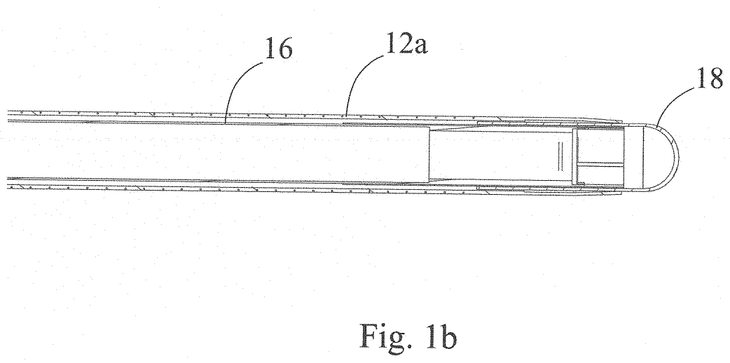

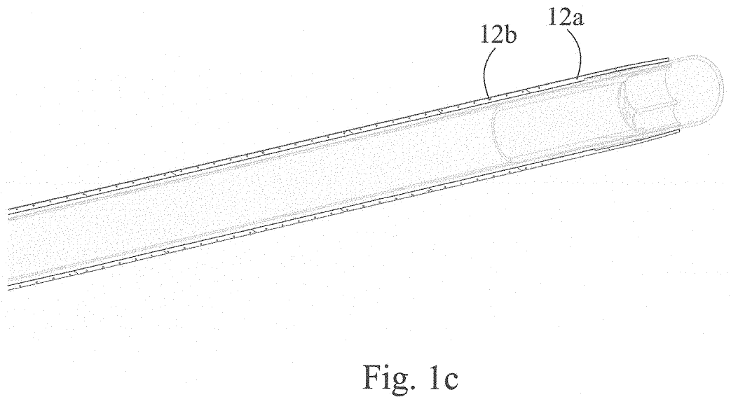

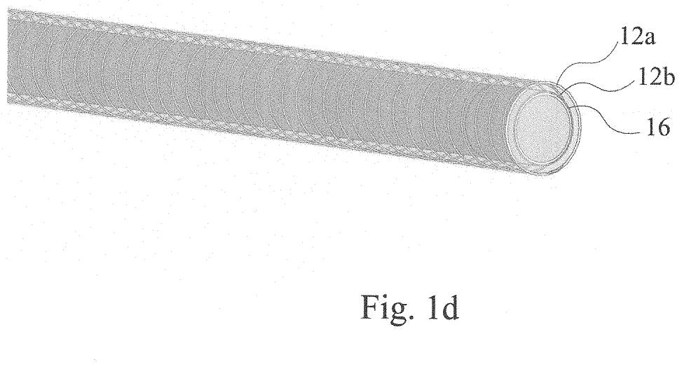

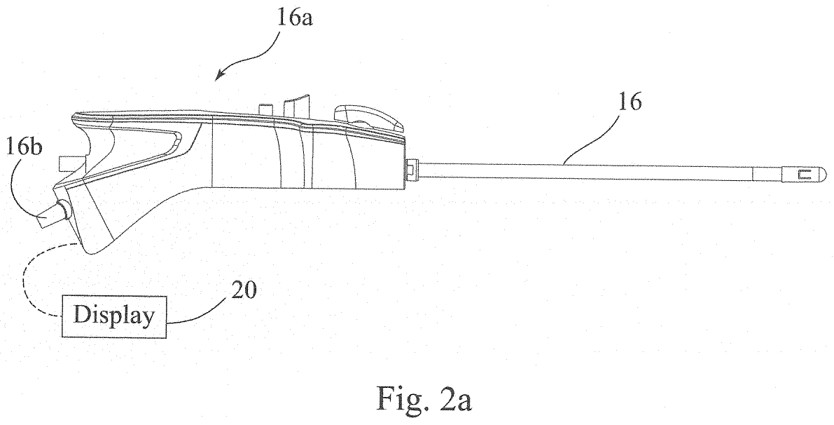

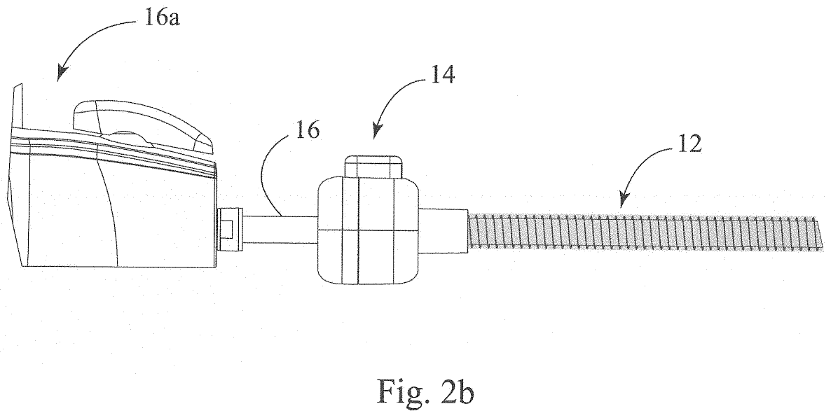

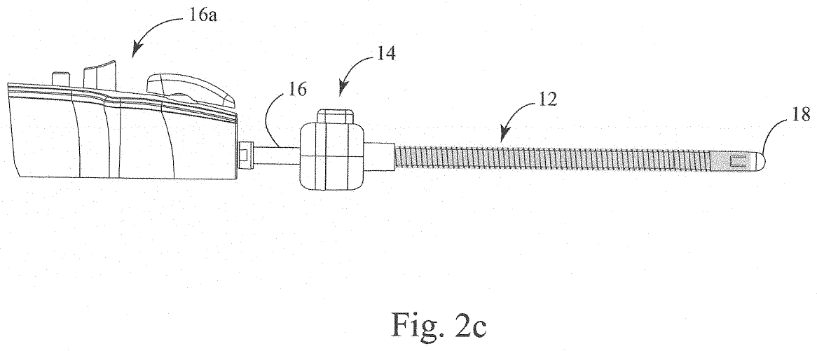

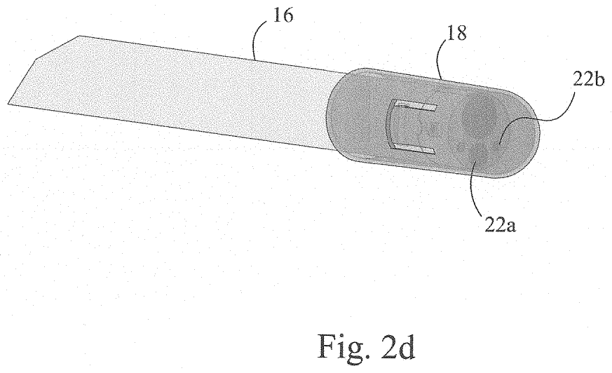

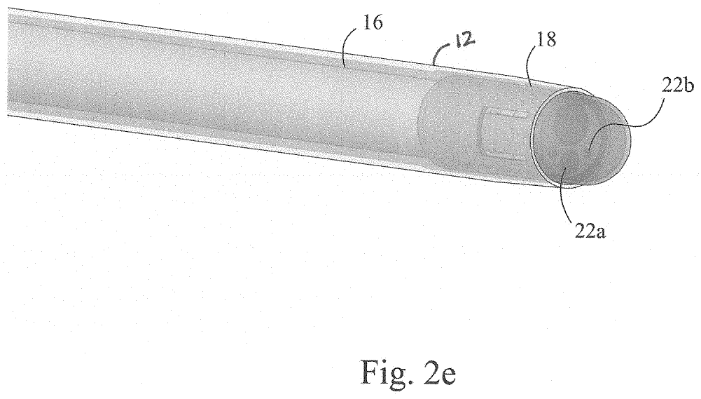

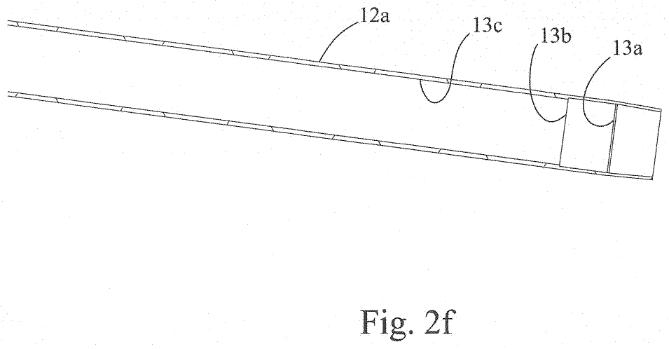

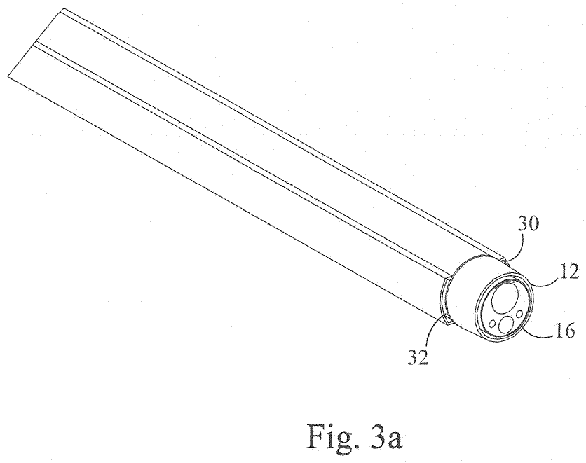

Referring to FIGS. 1a-1d, a medical device 10 according to some embodiments comprises an introducer 12 extending distally from a handle or head 14. A viewing endoscope comprises a handle 16a (FIG. 2a) and a cannula 16 can extend inside introducer 12 and has a distal end that houses a video camera and a light source and a proximal portion that extends proximally from the introducer 12 when cannula 16 is in the introducer. The distal end of the cannula 16 is at or protrudes distally from the distal end of introducer 12 when cannula 16 is in the introducer. The introducer 12 has open proximal and distal ends but its distal end is covered with an optical cap 18 that protrudes distally from the distal end of introducer 12. The distal end of introducer 12 is beveled such that its external diameter decreases toward the distal end of introducer 12 and approaches its internal diameter where introducer 12 terminates in the distal direction. Optical cap 18 has a hollow proximal portion with an outside diameter that matches the inside diameter of introducer 12 such that, as seen in FIG. 1b, there is a smooth transition between the outside of optical cap 18 and the outside of introducer 12, and there is little or no space between the inside of the distal end of introducer 12 and the outside of the proximal portion of optical cap 18. Optical cap 18 is releasably secured to the distal end of endoscope cannula 16. As one example, optical cap 18 is secured to the distal end of cannula 16 by a friction fit. As another example, optical cap 18 has a clip-like inward protrusion at its inside wall that fits in a matching depression or catch at the distal end of endoscope cannula 16 to help keep the cap in place while cannula 16 is being pushed distally into or withdrawn proximally from introducer or sheath 12. Optical cap 18 can be detached from cannula 16 after the cannula has been pulled out of introducer or sheath 12.

Optical cap 18 is made of a material that is sufficiently transparent to allow the camera at the distal end of endoscope cannula 16 to take clinical quality images and is shaped to reduce image distortion. A suitable material for optical cap 18 is polycarbonate but other material with suitable optical and material properties can be used. An example of wall thickness at the proximal portion of optical cap 18 is 0.3 mm, but wall thickness can range from 0.2 to 1.0 mm depending on the size of the distal end of endoscope cannula 16 and the inside diameter of introducer tube 12a (FIG. 1b). The distal end of optical cap 18 preferably is within 8 mm of the distal end of endoscope cannula 16 but this distance can range from 3 to 25 mm. The distal portion of optical cap 18 preferably is rounded, for example to a radius of curvature ranging from 2.5 to 5 mm.

Introducer or sheath 12 comprises a hollow tube 12a. In different examples discussed below, the wall of tube 12a can be (a) a flexible tubular plastic material that can be in a single layer or multiple layers and an internal coil 12b of a different material, such a coil of a metal wire or strip, that stiffens the plastic material while still allowing at least a portion thereof to bend while tube 12a is being introduced or has been introduced into a passageway, with the internal coil 12b extending over the entire length of tube 12a or preferably extending over some of the length of tube 12a only, and (b) multiple layers of material, without coil reinforcement. In all cases, the rigidity and flexibility of tube 12a can be the same over the entire length of tube 12a or can vary along the tube length.

In embodiments in which tube 12a is wire-reinforced, as seen in FIG. 1d, coil 12b can be embedded in the plastic material wall of tube 12a or can be between layers of plastic material. While wire coil 12b can extend over the entire length of tube 12a, but preferably wire coil 12b extends over some of the length of tube 12a only, for example from the proximal end of tube 12a to several cm from the distal end of tube 12a. In this manner, a distal portion of tube 12a can be less rigid and more flexible than the remainder of tube 12a. This can be particularly desirable in brain treatment procedures, where a tube 12a with a more flexible distal portion can help reduce trauma to tissue as tube 12a is being pushed to its desired position in the patient's cranium. In other examples, wire coil 12b can extend over an intermediate portion of tube 12a, offset by several cm from each of the proximal and distal ends of tube 12a. In still other examples, wire coil 12b can be in two or more sections separated from each other along the length of tube 12a, to thereby make less stiff and more flexible selected portions of tube 12a that are spaced from each other along the length of tube 12a.

One example of a plastic material suitable for the wall of introducer tube 12a is polyurethane. Other examples include polyvinyl Chloride and Pebax. Reduced rigidity and increased flexibility of one or more portions of tube 12a can be further facilitated by reducing the wall thickness of tube 12a at a distal portion thereof or at other selected portions thereof. Such reduction of wall thickness can be done with or without having the coil 12b extend into the distal portion of tube 12a.

In embodiments that do not use wire coil reinforcements, tube 12a preferably is multilayer, having two or more layers of material such as PTFE, FEP, or polyurethane and pebax; although it can be a single layer. FIG. 2f illustrates an example of a 3-layer wall of tube 12a, consisting of layers 13a, 13b and 13c. Omitting wire coil 12b can provide more flexibility, or can increase the inside diameter of tube 12a without increasing its outside diameter, to thereby provide a wider channel for surgical or viewing devices compared to an introducer with a coil 12b. When multilayer, the materials of the different layers can be the same in all respects or can differ from each other by wall thickness and/or by the nature of the layer material, to thereby affect the stiffness and flexibility of tube 12a or of selected longitudinal portions of the tube 12a. For example, when less rigidity and more flexibility are desired at a distal portion of tube 12a, for example over a few cm from the distal end of the tube 12a, the distal portion of tube 12a can be made with fewer layers of material or by reducing the thickness of one or more of the layers. When tube 12a is a single layer of plastic material, the wall thickness can be reduced at a distal portion thereof to affect stiffness and flexibility.

The outside diameter of tube 12a can be 8 mm but can range from 4 to 12 mm to match different needs and applications. An example of wall thickness of tube 12a is 0.5 mm but can range from 0.4 mm to 0.8 mm depending on the application. The wire coil material can be stainless steel, with wire thickness of 0.15 mm in one example and a range of 0.1 to 0.5 mm in other examples.

Introducer 12, comprising tube 12a (and coil 12b when a wire coil is used) preferably has a combination of stiffness and flexibility configured for a desired medical procedure. For example, when introducer 12 is intended for insertion along a straight path in a patient, higher stiffness and less flexibility are chosen for the material and wall thickness of tube 12a and for the material and wire gauge of coil 12b when a wire coil 12b is used. When introducer 12 is intended for insertion along a curved path in a patient, or needs to be bent after insertion, for at least some portions along the length of tube 12a less stiffness and more flexibility are chosen for the material and wall thickness of tube 12a and for the wire gauge of coil 12b when a coil is used. Introducer 12 can be made to deviate from a straight longitudinal axis by inserting therein the cannula 16 of a viewing endoscope that has a pre-formed curvature, or an endoscope cannula that can be curved by controls at an endoscope handle. In some embodiments, curves can be pre-formed in introducer tube 12a, and endoscope cannula 16 can be made sufficiently flexible to conform to such curves.

FIG. 1d shows a cross-section through an intermediate portion of introducer tube 12a with an endoscope cannula 16 therein according to some embodiments. Introducer tube 12a, which can be multilayer or a single layer and has a stiffening wire coil 12b that can be omitted for certain applications. Endoscope cannula 16 is shown in the center, smaller in outside diameter than the inside diameter of tube 12a to allow convenient insertion and withdrawal.

It can be challenging for a surgeon to introduce tube 12a into tissue, especially brain tissue, with minimal or no trauma. Selecting stiffness and flexibility at appropriate portions of tube 12a, and being able to view in real time images on display 20 as tube 12a is being pushed into the patient and possibly is changing shape and bending in the process, can be particularly efficacious. Selecting the proper materials with suitable thickness, and a distal portion of tube 12a that can be easily deformed under certain pressure and visualizing the progress and changes in bending of tube 12a can improve the outcome of a surgical procedure.

FIGS. 2a-2f illustrate the device of FIGS. 1a-1d according to some embodiments and additionally illustrate further features of the device and assist in describing a process of using the device. FIG. 2a illustrates a viewing endoscope that has a cannula 16 and a handle 16a with ports such as 16b for fluids and suction connected to respective suction and fluid ports at the distal tip of cannula 16. The viewing endoscope can be as described in commonly assigned U.S. Pat. Nos. 9,968,249 and 10,441,153, which are hereby incorporated by reference. Another viewing endoscope can be used instead that has a cannula with a video camera 22a and a light source 22b at its distal end. The camera and light source are illustrated at 22a and 22b, respectively, in FIG. 2d. As an initial step, optical cap 18 is secured to the distal end of cannula 16 as seen in FIG. 2d. FIG. 2c illustrates introducer 12 with cannula 16 inserted therein such that optical cap 18 protrudes distally from the distal end of introducer 12, as seen in FIG. 2e. In the configuration illustrated in FIG. 2c, a surgeon or other health practitioner inserts the medical device 10 (the introducer/endoscope/cap unit) into a patient while observing its progress on a display 20 (FIG. 2a) that receives real time images such as video from camera 22a at the distal end of endoscope cannula 16. The camera 22a and the light source 22b are described in more detail in the patents incorporated by reference. After the introducer/cannula/cap unit 10 has reached a desired depth in the patient, endoscope cannula 16 together with the optical cap 18 is withdrawn while introducer tube 12a stays in place in the patient. Now the surgeon can insert surgical instrument or other devices through introducer tube 12a. Viewing endoscope cannula 16 can be put back into introducer tube 12a without optical cap 18 so that ports at the distal tip of cannula 16 can be used, for example for suction from the patient or for irrigation as described in the patents incorporated by reference. Other surgical instruments can be inserted instead of cannula 16, such as pincers, abraders, cauterizers, and others. The surgical instruments can be inserted into a patient cavity or tissue without an obturator or dilator. In other words, the introducer 12 and the cannula 16 are configured for introduction as a unit into a patient cavity or tissue. Thus, it is possible to avoid undesired injury to tissues of patients when inserting surgical instruments.

In some embodiments, the method described above is used to access and perform a surgical procedure on a lesion in a patient's brain. After introducer 12 is placed with its distal end positioned as desired relative to the lesion, the endoscope described in the commonly assigned patents incorporated by reference can be used to view and act on the lesion or other surgical instruments can be used.

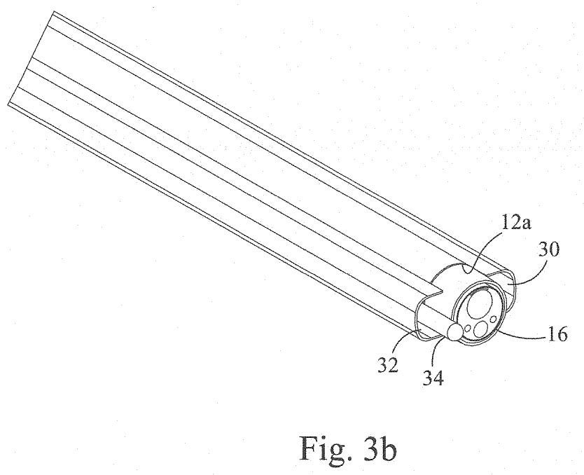

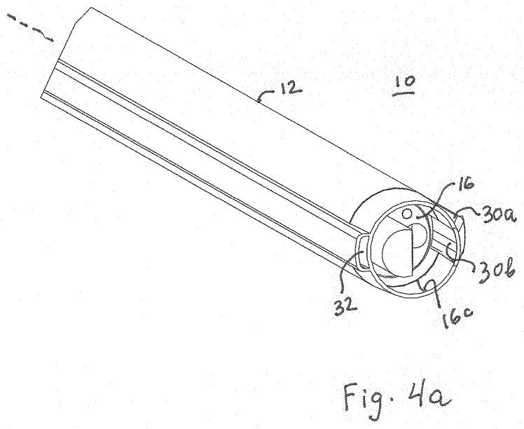

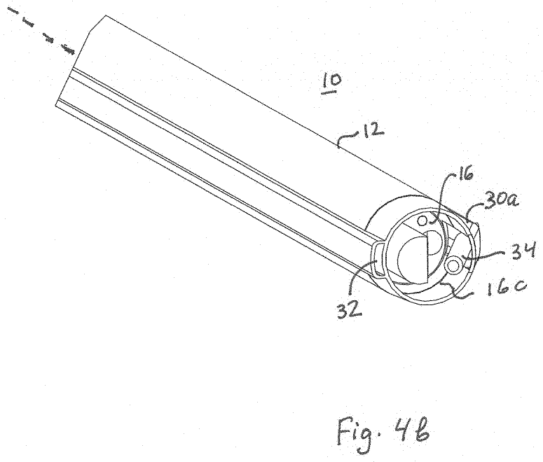

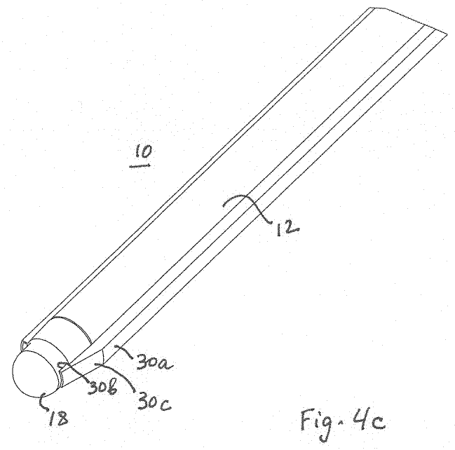

FIGS. 3a and 3b illustrate additional lumens 30 and 32 at an outside wall of tube 12a. In FIG. 3a, lumens 30 and 32 are collapsed and do not significantly increase the outside diameter of tube 12a. If there is no need to use lumens 30 and 32, they can retain the collapsed shape seen in FIG. 3a throughout the surgical procedure using tube 12a. However, one or both of lumens 30 and 32 can be utilized, for example after tube 12a has been introduced into the patient. As seen in FIG. 3b, a surgical tool 34 has been inserted into lumen 32 (and/or lumen 30) from a proximal end of the lumen 32 such that it protrudes from the distal end of lumen 32. In this manner, more complex surgical procedures can be carried out. For example, viewing endoscope cannula 16, with its camera 22a and the light source 22b can stay in place protruding from tube 12a, while a surgical tool 34 can be brought to tissue that is visualized with camera 22a, and possibly an additional tool can be brought to the tissue of interest through lumen 30. As another example, endoscope cannula 16 may have been withdrawn from tube 12a and replaced by a surgical tool and then, before or during a surgical procedure a viewing endoscope cannula 16 can be introduced through one of lumens 30 and 32 to visualize the tissue acted upon with the tool in tube 12a. In that case, an additional tool can be introduced through the lumen that is not occupied by endoscope cannula 16.

Although the foregoing has been described in some detail for purposes of clarity, it will be apparent that certain changes and modifications may be made without departing from the principles thereof. There can be many alternative ways of implementing both the processes and apparatuses described herein. Accordingly, the present embodiments are to be considered as illustrative and not restrictive, and the body of work described herein is not to be limited to the details given herein, which may be modified within the scope and equivalents of the appended claims.

For another embodiment, different types of side lumens can be used. The outlet of the side lumen can be directed toward the center line of the main lumen (for the cannula 16), so that a surgical tool 34 such as flexible surgical device or electro-surgical unit, can be directed toward the center line of the endoscope. This can be particularly useful for coagulation or biopsy. When working in a small space and handling multiple surgical devices with one hand, it is important for a surgeon to have easy access to a target.

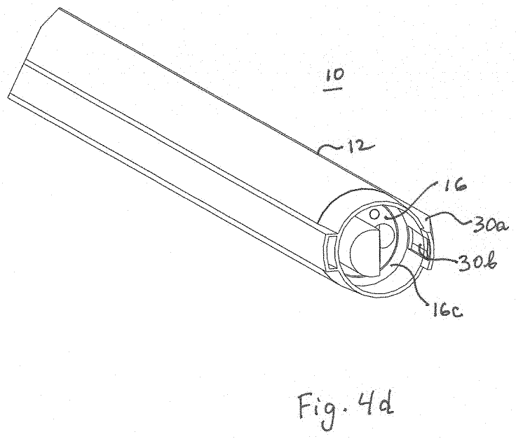

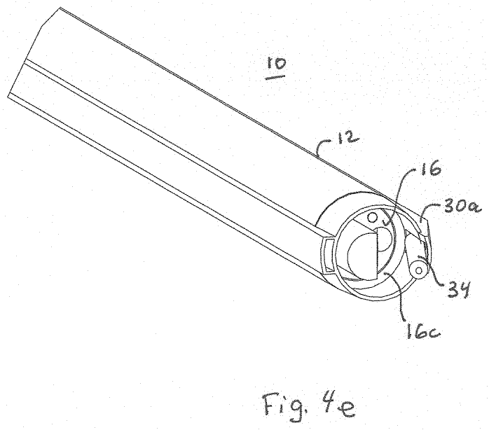

FIGS. 4a and 4b, FIGS. 4a and 4b illustrate an embodiment of the medical device 10 including a side lumen 30a before and after inserting the surgical tool 34. In particular, the medical device 10 includes an introducer 12, at least one lumen (30a and 32), and a cannula 16. As in the embodiments set forth above, the introducer 12 may include the coil 12a. The introducer 12 has a distal end and a proximal end (not illustrated) and the introducer 12 is open at the distal end and the proximal end. The cannula 16 is at least partially located inside the introducer 12 and has an outlet 16c. In the present embodiment, the at least one lumen includes a side lumen 30a and the lumen 32 respectively secured to outside of the introducer 12. In the present embodiment, the side lumen 30a has an outlet 30a facing toward a center (the dotted line in FIGS. 4a and 4b) of the cannula 16; in other words, the outlet 30a is formed on the inner surface of the introducer 12. As a non-limiting example, the outlet 30a of the side lumen 30a can be used to flush the outlet 16c of the cannula 16 with appropriate fluid.

In addition, through the design of the side lumen 30a having the outlet 30b extending toward the center of the cannula 16 (the dotted line in FIGS. 4a and 4b), a surgical tool 34 such as flexible surgical device or electro-surgical unit can be directed toward the center (the dotted line in FIGS. 4a and 4b) of the cannula 16 of the endoscope through the outlet 30b. For example, the surgical tool 34 may include devices for coagulation or biopsy. As noted, when working in a small space and handling multiple surgical devices with one hand, it is important for a surgeon to have easy access to a target.

In the present embodiment, the side lumen 30a and the lumen 32 are locates at opposite sides of the introducer 12; however, in another embodiment the locations of the side lumen 30a and the lumen 32 can be reversed or the side lumen 30a can be located at both sides instead of the lumen 32 as needed or desired for a specific surgical procedure. In addition, the lumen 32 can be omitted if not needed or desired for a particular surgical procedure.

FIG. 4c-4e illustrate another embodiment of the medical device 10, with a different design of the side lumen 30a. FIG. 4c shows the medical device 10 with the optical cap 18 configured to cover the outlet 30b of the side lumen 30a. The optical cap 18 can be configured to cover the outlet 30b of the side lumen 30a. The side lumen 30a can include an inclined surface 30c at the end which is close to the optical cap 18. With the disposition of the inclined surface 30c, the possible drag exerted on the side lumen 30a may be reduced while the introducer 12 is moving through a passageway.

FIGS. 4d and 4e illustrate the medical device 10 before and after inserting the surgical tool 34. The difference between the present embodiment and the previous embodiment (FIG. 4a-4b) is that the side lumen 30a and the outlet 30b of the side lumen 30a are configured to extend over the distal end (or the outlet 16c) of the cannula 16. Through the designs of the outlet 30b of the side lumen 30a, the surgical tool 34 can be extended further than in the previous embodiment illustrated in FIG. 4b, to provide better visualization by reducing the ratio of its footprint. In addition, possible tissue damage during insertion process can also be prevented or at least reduced.

It should be understood that features of one of the described embodiments can be used in another of the embodiments within the scope of the disclosure of this patent specification.

1. A method of endoscopic intervention using a dilator-less and obturator-less introducer, comprising:
securing an opening at a proximal end of an optical cap to a distal end of an endoscope cannula that has distally facing camera and light source, and intermediate portion and a proximal portion;
inserting the distal end of the endoscope cannula, with said optical cap secured thereto, in the distal direction into a tubular introducer that has open proximal and distal ends such that:
a distal end of the optical cap protrudes distally from the distal end of the introducer;
an outside wall of the proximal end of said optical cap and an outside wall of the distal end of the introducer are dimensioned to form a smooth transition from the introducer to the optical cap; and
the proximal portion of the endoscope cannula extends proximally from the proximal end of the introducer and the intermediate portion of the endoscope cannula is between the proximal and distal ends of the introducer;
inserting the introducer, with the intermediate portion of the endoscope cannula therein and with said optical cap secured to the distal end of the cannula, in the distal direction into a passageway or tissue, using the rounded distal end of said optical cap to dilate the passageway or form a passageway in tissue, while observing at a display images taken by said camera at the endoscope distal end through said optical cap;
withdrawing the endoscope cannula together with the optical cap from the introducer in the proximal direction; and
inserting one or more surgical instruments or devices distally into the introducer to act on tissue adjacent the distal end of the introducer.

2. The method of claim 1, in which the step of inserting the introducer comprises inserting an introducer made of a flexible plastic material reinforced with a metal coil.

3. The method of claim 2, in which the step of inserting the introducer comprises inserting an introducer in which a distal portion of the introducer is free of said metal coil and is less rigid and more flexible than another portion of the introducer.

4. The method of claim 1, in which the step of inserting the endoscope cannula into the introducer and/or the step of inserting the introducer into the passageway or tissue of the patient comprises selectively bending the introducer to deviate from a straight longitudinal axis.

5. The method of claim 4, further comprising making at least a selected portion of a length of the introducer flexible and introducing a bent endoscope cannula therein to thus bend the introducer.

6. The method of claim 1, in which the step of inserting the introducer comprises inserting an introducer with a distal end that is beveled such that the diameter of its outside wall approaches the diameter of its inside wall toward a distal tip of said introducer.

7. The method of claim 1, in which the step of inserting an introducer comprises inserting an introducer having one or more collapsed lumens at an outside wall of the introducer.

8. The method of claim 7 further comprising the step of introducing an implement into and thereby expanding one or more of said one or more lumens.

9. A medical device comprising:
a tubular introducer having open proximal and distal ends;
an endoscope cannula having a proximal end extending proximally from said introducer, an intermediate portion within said introducer, and a distal end at or protruding distally from the distal end of the introducer;
said endoscope cannula having a camera and a light source at its distal end;
an optical cap removably secured to said distal end of the endoscope cannula and having a rounded distal portion and a hollow proximal portion into which the distal end of the endoscope cannula is inserted;
wherein:
said rounded distal portion of the optical cap protrudes distally from the distal end of the introducer; and
a distal portion of the introducer has an outside dimension that decreases toward the distal end of the introducer and the proximal portion of the optical cap has an outside dimension that matches an inside diameter of the distal end of the introducer to thereby provide a smooth transition between the outside of the optical cap and the outside of the distal end of the introducer; and
an image display coupled to said camera to receive and display images taken with said camera through said optical cap;
said introducer and cannula therein being configured for introduction as a unit into a patient cavity or tissue without an obturator or dilator.

10. The medical device of claim 9, in which said introducer is made of a flexible tubular plastic material and an internal coil of a different material stiffening the tubular plastic material while still allowing at least a distal portion thereof to bend while or after being introduced into said passageway or tissue.

11. The medical device of claim 9, in which said endoscope cannula includes a bend in its intermediate portion and said introducer is configured to bend to match the bend in the endoscope cannula.

12. The medical device of claim 9, further including an endoscope handle from which said endoscope cannula extends distally, wherein said cannula is configured to bend at an intermediate portion under operator control applied at the endoscope handle, while the endoscope cannula is in the introducer.

13. The medical device of claim 9, in which, optical cap, and introducer are configured for withdrawal of the endoscope together with the optical cap from the introducer in the proximal direction.

14. The medical device of claim 9, in which said optical cap is made of an optically transparent material.

15. A device comprising:
a tubular introducer open at proximal and distal ends thereof;
an elongated cannula having a camera and a light source at a distal end thereof and further having an intermediate portion that is inside said introducer, a proximal portion extending proximally from the introducer, and a distal portion;
an optical cap secured to said distal portion of the cannula, over said camera and light source, and having a rounded end extending distally from the distal end of the introducer;
wherein said cap and introducer are dimensioned to form a smooth transition from the distal end of the introducer to a proximal portion of the cap that extends distally from the introducer; and
a display operatively coupled with said camera at the distal end of the cannula to receive images taken with the camera and display said images;
said device being configured for insertion of the introducer, cannula, and optical cap into a passageway or tissue without using a dilator or obturator to form or dilate the passageway or tissue.

16. The device of claim 15, in which a distal portion of the introducer is more flexible than another portion of the introducer to thereby facilitate bending said distal portion as the introducer is being moved into a passageway or tissue.

17. The device of claim 15, in which at least a portion of said introducer is made of a plastic material reinforced with a metal coil.

18. The device of claim 15, in which at least a portion of said introducer is made of plural layers of plastic material configured to make a distal portion of the introducer less rigid and more flexible than another portion of the introducer.

19. The device of claim 15, further including at least one collapsed lumen secured to the outside of the introducer.

20. The device of claim 15, in which one of the optical cap and the distal portion of the cannula includes a clip and the other includes a catch, configured for the clip to engage the catch and thereby secure the cap to the cannula.

21. The device of claim 19, wherein the at least one lumen includes a side lumen having an outlet facing toward a center of the cannula.

22. The device of claim 19, the outlet of the side lumen is formed on an inner surface of the cannula.

23. The device of claim 21, wherein the outlet of the side lumen extends over the distal end of the cannula.

24. The device of claim 23, wherein the optical cap is configured to cover the outlet of the side lumen.

25. A medical device, comprising:
an introducer having a distal end and a proximal end, the introducer opened at the distal end and the proximal end;
at least one lumen secured to outside of the introducer; and
a cannula having a distal end and at least partially located inside the introducer;
wherein, the at least one lumen includes a side lumen having an outlet facing toward a center of the cannula.

26. The medical device of claim 25, wherein the outlet of the side lumen extends over the distal end of the cannula.

27. The medical device of claim 26, further comprising an optical cap configured to cover the outlet of the side lumen.

28. The device of claim 25, the outlet of the side lumen is formed on an inner surface of the cannula.
